(19) United States
(12) Patent Application Publication
(10) Pub. No.: US 2025/0025258 A1
(43) Pub. Date: Jan. 23, 2025

(54) BINOCULAR SURGICAL LOUPES WITH LED HEADLIGHT AND HEARING PROTECTION

(71) Applicant: Jennifer DiPiero, Peninsula, OH (US)

(72) Inventor: Jennifer DiPiero, Peninsula, OH (US)

(21) Appl. No.: 18/357,103

(22) Filed: Jul. 22, 2023

(51) Int. Cl. A61B 90/35; A61B 90/00; G02B 25/00; G02C 7/08

(52) U.S. Cl. CPC A61B 90/35; A61B 90/361; G02B 25/004; G02C 7/088; A61B 2090/3616

(57) ABSTRACT

A binocular dental surgical loupes with two transparent lenses mounted in a frame and joined together by the frame. A front guard portion between the two transparent lenses including a bridge portion for supporting the frame on the nose of a user. The frame includes front portions which are interconnected by the bridge portion and surround and hold the two transparent lenses in place. A pair of temples are mounted by hinge sections to the front portions. A pair of dental loupes disposed in front of the two transparent lenses. A light mounted to the frame and hearing protection comprising first and second earplugs mounted to the frame.

### TECHNICAL FIELD OF THE INVENTION

The present invention relates generally to dental loupes with a headlight and hearing protection and more particularly to binocular dental surgical loupes with an LED headlight and hearing protection.

### BACKGROUND OF THE INVENTION

A dental diagnosis of the oral cavity often requires a headlight in combination with magnifying glasses that enables the dentist or technician to clearly see the state of the teeth and gums.

Headlights used for various diagnoses can be a light source simply attached to a head mounting means, or a goggle or glasses with a light source attached to a frame portion. These headlights are used by being mounted to the doctors or technicians head, so that the field of the light is set to face the line of sight when attached to the head. During diagnosis work, the light source brightly illuminates the part to be diagnosed, and there is no need to make adjustments each time a different section is illuminated. Since both hands of the operator are free, the work will be done very quickly and smoothly. It is often desired to protect the eyes of an operator from being injured by flying debris in addition to protecting the hearing of the user from being injured by excessive noise.

Solutions for these problem include providing separate safety glasses and earplugs. A problem occurs when a user can use only one protection device when both are needed for proper protection in the work environment. The problem with these solutions is that the user can wear the safety glasses without using the earplugs, thus, causing damage to his or her hearing or the user can wear the earplugs without the safety glasses thus potentially causing damage to his or her vision.

### SUMMARY OF THE INVENTION

According to another embodiment of the present invention, there is disclosed a binocular dental surgical loupes with two transparent lenses mounted in a frame and joined together by the frame. A front guard portion between the two transparent lenses including a bridge portion for supporting the frame on the nose of a user. The frame includes front portions which are interconnected by the bridge portion and surround and hold the two transparent lenses in place. A pair of temples are mounted by hinge sections to the front portions. A pair of dental loupes disposed in front of the two transparent lenses. A light mounted to the frame and hearing protection comprising first and second earplugs mounted to the frame.

### BRIEF DESCRIPTION OF THE DRAWINGS

The structure, operation, and advantages of the present invention will become further apparent upon consideration of the following description taken in conjunction with the accompanying FIGURES (FIGS.). The figures are intended to be illustrative, not limiting. Certain elements in some of the FIGURES may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

FIG. 1 is a side, three-dimensional view of a binocular dental surgical loupes with an LED headlight and hearing protection, in accordance with the present invention.

### DETAILED DESCRIPTION OF THE PREFERRED EMBODIMENTS

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

In the drawings accompanying the description that follows, often both reference numerals and legends (labels, text descriptions) will be used to identify elements. If legends are provided, they are intended merely as an aid to the reader, and should not in any way be interpreted as limiting.

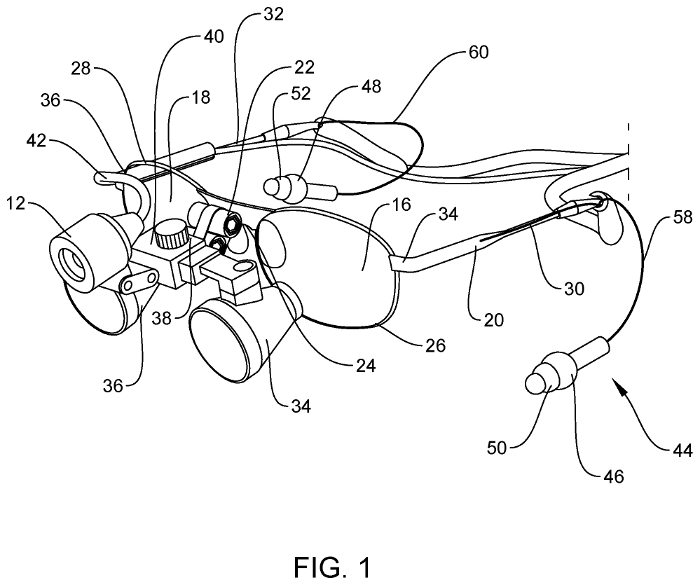

Referring to FIG. 1 there is illustrated a side, three-dimensional view of a binocular dental surgical loupes 10 with an LED headlight 12 and hearing protection 14, in accordance with the present invention.

Referring to FIG. 1, the binocular dental surgical loupes 10 comprise two transparent lenses 16 and 18 mounted in a frame 20 and joined together by the frame. A front guard portion 22 between the two transparent lenses 16 and 18 includes a well known bridge portion 24 for supporting the frame on the nose of the user. The front guard portion 22 preferably includes an elongated resilient member secured to the bridge portion 24 and disposed adjacent and between the two transparent lenses 16 and 18. The elongated resilient member is preferably constructed in the shape of a curve and is secured to the bridge portion 24.

The frame 20 includes the front portions 26 and 28 which are interconnected by the bridge portion 24 and surround and hold the two transparent lenses 16 and 18 in place. A pair of temples 30 and 32 are mounted by hinge sections 34 and 36 to the front portions 26 and 28.

Dental loupes 34 and 36 are placed in front of the two transparent lenses 16 and 18. The dental loupes 34 and 36 can be mounted directly to the lenses 16 and 18, or secured to a frame 38 which can be mounted to the guard portion 22 of the frame so that the dental loupes 34 and 36 can flip up and down in front of the lenses. This results in the dental loupes being adjustable, flexible and adaptable.

An ultra-mini light 12 can be mounted to the frame with a mounting clamp 40 with the ability to pivot so that the light can be adjusted with respect to the loupes 34 and 36. The ultra-mini light 12 can be connected to a battery through a wire 42 or alternatively be wireless.

Referring again to FIG. 1, there is illustrated hearing protection 44 comprising first and second earplugs 46 and 48 which are mounted to the pair of temples 30 and 32, respectively. The earplugs have multiple curved elements 50 and 52 which snugly engage an adjacent auditory canal. The earplugs 46 and 48 preferably further includes an attachment stem 54 and 56, respectively, for attaching the earplugs 46 and 48 to the binocular dental surgical loupes 10 with an LED headlight 12 and hearing protection 14. Eyesight and hearing safety apparatus 20. Each earplug 46 and 48 is preferably constructed from well known rubber material. Elongated resilient members 58 and 60 attach at one end to the earplugs and at the other end to the pair of temples 30 and 32. The length of the resilient members 58 and 60 are selected so that the earplugs can easily be inserted in an adjacent ear canal.

In a preferred embodiment, the earplugs 46 and 48 are provided with a replaceable soft tip, electronics with integrated multi-band automatic amplitude control, and active noise attenuation without feedback noise cancellation. Each of the earplugs 46 and 48 preferably fit at least partially in an ear canal, and do not interfere with glasses, long hair, or helmets.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

1. A binocular dental surgical loupes comprising:
two transparent lenses mounted in a frame and joined together by the frame;
a front guard portion between the two transparent lenses including a bridge portion for supporting the frame on the nose of a user;
the frame includes the front portions which are interconnected by the bridge portion and surround and hold the two transparent lenses in place;
a pair of temples are mounted by hinge sections to the front portions;
a pair of dental loupes disposed in front of the two transparent lenses;
a headlight mounted to the frame; and
hearing protection comprising first and second earplugs mounted to the frame.

2. The binocular dental loupes claim 1 wherein the two dental loupes are mounted directly to the lenses.

3. The binocular dental loupes of claim 1 wherein the two dental loupes are mounted to the front guard portion of the frame so that the dental loupes can flip up and down in front of the lenses.

4. The binocular dental loupes of claim 1 wherein the two dental loupes are pivotally mounted to the front guard portion of the frame so that the dental loupes can flip up and down in front of the lenses.

5. The binocular dental loupes of claim 4 wherein the headlight is mounted to the frame with a mounting clamp.

6. The binocular dental loupes of claim 5 wherein the headlight is mounted to the frame with a mounting clamp that the headlight can pivot and be adjusted with respect to the loupes.

7. The binocular dental loupes of claim 6 wherein the headlight is an ultra-mini light connected to a battery through a wire.

8. The binocular dental loupes of claim 6 wherein the headlight is a wireless ultra-mini-light.

9. The binocular dental loupes claim 5 wherein the hearing protection comprises first and second earplugs which are mounted to the pair of temples.

10. The binocular dental loupes of claim 9 wherein the earplugs have multiple curved elements which snugly engage an adjacent auditory canal.

11. The binocular dental loupes of claim 10 wherein the earplugs each include an attachment stem for attaching the earplugs to the binocular dental surgical loupes with an LED headlight and hearing protection.

12. The binocular dental loupes claim 11 wherein each earplug is constructed from rubber material.

13. The binocular dental loupes of claim 11 wherein elongated resilient members attach at one end to the earplugs and at an opposite end to the pair of temples.

14. The binocular dental loupes of claim 13 wherein the length of the resilient members are selected so that the earplugs can easily be inserted in an adjacent ear canal.

15. The binocular dental loupes of claim 14 wherein the earplugs are provided with a replaceable soft tip, electronics with integrated multi-band automatic amplitude control, and active noise attenuation without feedback noise cancellation.

16. The binocular dental loupes of claim 15 wherein the each of the earplugs fit at least partially in an ear canal.

17. The binocular dental loupes of claim 11 wherein the front guard portion includes an elongated resilient member secured to the bridge portion and disposed adjacent and between the two transparent lenses.

18. The binocular dental loupes of claim 17 wherein the elongated resilient member is constructed in the shape of a curve and is secured to the bridge portion.

19. A binocular dental surgical loupes 10 comprising:
two transparent lenses mounted in a frame and joined together by the frame;
a front guard portion between the two transparent lenses including a bridge portion for supporting the frame on the nose of a user;
the frame includes the front portions which are interconnected by the bridge portion and surround and hold the two transparent lenses in place;
a pair of temples are mounted by hinge sections to the front portions;
a pair of dental loupes disposed in front of the two transparent lenses;
a headlight mounted to the frame with a mounting clamp; and
hearing protection comprising first and second earplugs mounted to the pair of temples wherein the earplugs each include an attachment stem for attaching the earplugs to the binocular dental surgical loupes.

20. The binocular dental loupes of claim 19 wherein elongated resilient members attach at one end to the earplugs and at an opposite end to the pair of temples and wherein the length of the resilient members are selected so that the earplugs can easily be inserted in an adjacent ear canal.
